SEQUENCE LISTING (29 sequences)

SEQ ID NO: 1 - protein, 10 aa, Unknown
  Peptide Identified from the Hydrolysate of a Whey Protein
VLDTDYKYLL

SEQ ID NO: 2 - protein, 11 aa, Unknown
  Peptide Identified from the Hydrolysate of a Whey Protein
TPPVDDEALEK

SEQ ID NO: 3 - protein, 5 aa, Unknown
  Peptide Identified from the Hydrolysate of a Whey Protein
VRTPT

SEQ ID NO: 4 - protein, 7 aa, Unknown
  Peptide Identified from the Hydrolysate of a Whey Protein
AASDISL

SEQ ID NO: 5 - protein, 7 aa, Unknown
  Peptide Identified from the Hydrolysate of a Whey Protein
DAQSAPL

SEQ ID NO: 6 - protein, 6 aa, Unknown
  Peptide Identified from the Hydrolysate of a Whey Protein
NENKVL

SEQ ID NO: 7 - protein, 10 aa, Unknown
  Peptide Identified from the Hydrolysate of a Whey Protein
DAQSAPLRVY

SEQ ID NO: 8 - protein, 8 aa, Unknown
  Peptide Identified from the Hydrolysate of a Whey Protein
LDAQSAPL

SEQ ID NO: 9 - protein, 5 aa, Unknown
  Peptide Identified from the Hydrolysate of a Whey Protein
KALPM

SEQ ID NO: 10 - protein, 6 aa, Unknown
  Peptide Identified from the Hydrolysate of a Whey Protein
IPAVFK

SEQ ID NO: 11 - protein, 6 aa, Unknown
  Peptide Identified from the Hydrolysate of a Whey Protein
GLDIQK

SEQ ID NO: 12 - protein, 13 aa, Unknown
  Peptide Identified from the Hydrolysate of a Whey Protein
PTPEGDLEILLQK

SEQ ID NO: 13 - protein, 5 aa, Unknown
  Peptide Identified from the Hydrolysate of a Whey Protein
IIAEK

SEQ ID NO: 14 - protein, 10 aa, Unknown
  Peptide Identified from the Hydrolysate of a Whey Protein
VLVLDTDYKK

SEQ ID NO: 15 - protein, 7 aa, Unknown
  Peptide Identified from the Hydrolysate of a Whey Protein
ALPMHIR

SEQ ID NO: 16 - protein, 6 aa, Unknown
  Peptide Identified from the Hydrolysate of a Whey Protein
LDTDYK

SEQ ID NO: 17 - protein, 4 aa, Unknown
  Peptide Identified from the Hydrolysate of a Whey Protein
LQKW

SEQ ID NO: 18 - protein, 6 aa, Unknown
  Peptide Identified from the Hydrolysate of a Whey Protein
VAGTWY

SEQ ID NO: 19 - protein, 7 aa, Unknown
  Peptide Identified from the Hydrolysate of a Whey Protein
LNENKVL

SEQ ID NO: 20 - protein, 9 aa, Unknown
  Peptide Identified from the Hydrolysate of a Whey Protein
VEELKPTPE

SEQ ID NO: 21 - protein, 7 aa, Unknown
  Peptide Identified from the Hydrolysate of a Whey Protein
VAGTWYS

SEQ ID NO: 22 - protein, 5 aa, Unknown
  Peptide Identified from the Hydrolysate of a Whey Protein
HIRLS

SEQ ID NO: 23 - protein, 7 aa, Unknown
  Peptide Identified from the Hydrolysate of a Whey Protein
DTDYKKY

SEQ ID NO: 24 - protein, 9 aa, Unknown
  Peptide Identified from the Hydrolysate of a Whey Protein
NSAEPEQSL

SEQ ID NO: 25 - protein, 7 aa, Unknown
  Peptide Identified from the Hydrolysate of a Whey Protein
SAPLRVY

SEQ ID NO: 26 - protein, 10 aa, Unknown
  Peptide Identified from the Hydrolysate of a Whey Protein
LDAQSAPLRV

SEQ ID NO: 27 - protein, 10 aa, Unknown
  Peptide Identified from the Hydrolysate of a Whey Protein
TPPVDDEALE

SEQ ID NO: 28 - protein, 7 aa, Unknown
  Peptide Identified from the Hydrolysate of a Whey Protein
TKIPAVF

SEQ ID NO: 29 - protein, 8 aa, Unknown
  Peptide Identified from the Hydrolysate of a Whey Protein
LKPTPEGD

(19) United States
(12) Patent Application Publication
(10) Pub. No.: US 2014/0011742 A1
(43) Pub. Date: Jan. 9, 2014

(54) Bioactive Peptides and Proteins Containing Bioactive Peptides, their Uses and Processes for Making the Same

(71) Applicants: Jacqueline Sedman, Kirkland (CA); Ashraf A. Ismail, Westmount (CA); Ahmed I. Gomaa, Montreal (CA)

(72) Inventors: Jacqueline Sedman, Kirkland (CA); Ashraf A. Ismail, Westmount (CA); Ahmed I. Gomaa, Montreal (CA)

(73) Assignee: MCGILL UNIVERSITY, Montreal, QC (CA)

(21) Appl. No.: 13/992,685

(22) PCT Filed: Dec. 8, 2011
(86) PCT No.: PCT/CA2011/001346

§ 371 Date: Sep. 23, 2013

Related U.S. Application Data
(60) Provisional application No. 61/421,441, filed on Dec. 9, 2010.

(51) Int. Cl. C07K 14/47; C07K 14/415

(52) U.S. Cl. CPC C07K 14/47; C07K 14/415
USPC 514/15.7; 530/350; 435/68.1; 530/378; 530/365; 514/16.3

(57) ABSTRACT

A process for treating a protein before hydrolytic digestion, the process comprising exposing the protein to at least one cycle of microwave irradiation to produce a microwave treated protein containing one or more bioactive peptides. Further hydrolyzing the microwave treated protein to release at least one of the one or more bioactive peptides. A pharmaceutical composition, supplement and food product including the microwave treated protein or the one or more released bioactive peptides.

### FIELD OF THE INVENTION

The present invention relates generally to bioactive peptides and proteins containing bioactive peptides, their uses and processes for making the same. Specifically, but not exclusively, the present invention relates to bioactive peptides and proteins containing bioactive peptides produced by a microwave treatment and having medicinal and/or nutraceutical benefits.

### BACKGROUND OF THE INVENTION

Proteins are added to foods because of their functional properties or to enhance nutritional and health qualities of a food product. Bioactive proteins and peptides derived from many food proteins are known to have a positive impact on the cardiovascular, immune, nervous and gastrointestinal systems of users. For example, many proteins and peptides have anti-hypertensive properties, anthithrombotic effects, opioid activities, immunomodulatory activities, mineral sequestering properties, and antioxidant and antimicrobial activities. Some proteins play a role in reducing the risk of coronary heart disease by lowering plasma cholesterol and triglycerides while others can aid in controlling insulin fluctuations. Protein ingredients are available as isolates, concentrates, and hydrolysates.

Bovine, ovine and caprine milk and eggs are an important source of protein and bioactive peptides in human diets, as well as fish and plants such as soy beans, peas, chickpeas, flax, brown rice, corn, wheat, oats and potatoes.

Some proteins found, for example, in milk, soy and peas have positive effects in the areas of satiety, weight management and sustained energy. These effects are likely due to the slow digestion of proteins which prolongs the feeling of fullness. Whey proteins such as alpha-lactalbumin and bovine serum albumin have been researched extensively in the prevention and treatment of cancer. Whey protein supplementation has also shown benefits in exercise performance and enhancement.

The whey protein fraction from milk proteins is a source of amino acids and contains several bioactive health promoting proteins such as the α-lactalbumin and β-lactoglobulin which have been shown to contain bioactive peptides. For example, peptides showing opioid and angiotensin I-converting enzyme inhibitory activity have been derived from the hydrolysis of α-lactalbumin and β-lactoglobulin. Angiotensin I-converting enzyme (ACE, peptidyl dipeptide hydrolase, EC 3.4.15.1) has been classically associated with the renin-angiotensin system, which regulates peripheral blood pressure.

Therefore, there is a need for the identification of new proteins containing bioactive peptides or new bioactive peptides. There is also a need for proteins and/or bioactive peptides having enhanced nutraceutical, medicinal and/or health benefits. There is also a need for improved processes for isolating, releasing or generating bioactive peptides from proteins.

### SUMMARY OF THE INVENTION

Accordingly, aspects of the present invention provide a new process for releasing, isolating or generating bioactive peptides from proteins, as well as for pre-treating a protein before the bioactive peptide is released, isolated or generated. Aspects of the present invention also provide proteins containing new bioactive peptides. By means of the new process, the functional, medicinal and/or nutraceutical properties of the proteins and/or the released, isolated or generated bioactive peptides are enhanced. By “released peptides” is meant peptides that have been cleaved, prepared or generated from their parent proteins by any method.

From a first aspect, there is provided a process for treating a protein before hydrolytic digestion (‘pre-treatment’), the process comprising exposing the protein to microwave irradiation to produce a microwave treated protein containing one or more bioactive peptides. This pre-treatment can be followed by hydrolytic digestion of the microwave treated protein with at least one proteolytic enzyme to release, isolate or generate at least one of the one or more bioactive peptides. The pre-treatment can also be followed by any other suitable method to release, isolate or generate at least one of the one or more bioactive peptides.

From another aspect, there is provided a process for releasing (preparing, isolating or generating) one or more bioactive peptides from a protein, the process comprising the steps of: a) exposing the protein in a solution to a microwave irradiation to produce a microwave treated protein; b) hydrolyzing the microwave treated protein with at least one proteolytic enzyme; and c) terminating said hydrolyzing step in order to release, isolate or generate one or more bioactive peptides.

Indeed, the inventors have demonstrated (see Examples) that surprisingly microwave treatment of whey and soy proteins prior to enzymatic hydrolysis yields pre-treated proteins containing hydrolysates or bioactive peptides that have enhanced nutraceutical and/or medicinal benefits. These pre-treated proteins can provide a non-pharmacological alternative for the prevention, control, modulation and/or treatment of diseases. It will be understood that whey and soy proteins were used as a model only and the present process is applicable to any type of proteins such as food proteins or proteins derived from plant or animal sources such as, but not limited to, soy, wheat, corn, milk, meat or egg. In addition, the inventors have demonstrated that an increase in the degree of hydrolysis of proteins coupled with an enhancement in ACE and antioxidant activities as a result of the microwave treatment of proteins (for example soy protein isolate or whey protein isolate) can be attributed to the formation of new bioactive peptides and/or an increase in bioactive peptide content in general.

Advantageously, the microwave irradiation of the protein can be performed as a pre-treatment or a pre-conditioning method before the hydrolytic digestion of a protein. The further step of hydrolytic digestion of the microwave treated protein can take place in vivo (for example when a mammal ingests the microwave treated protein) or in vitro. If in vitro, the proteolytic digestion or the hydrolyzing step can be carried out with at least one proteolytic enzyme. By virtue of the pre-treatment of protein by microwave irradiation before hydrolytic digestion, at least some of the properties or benefits of the protein or the subsequently isolated bioactive peptide(s), such as its ACE inhibition activity and hypertension modulation, are enhanced. Indeed, the microwave treatment or irradiation induces conformational changes to the proteins or food proteins or the proteins derived from food which can enhance, for example, their susceptibility to proteolytic enzymes in order to generate bioactive peptides. The inventors have demonstrated herein that the degree of hydrolysis of protein is enhanced when digestion takes place after microwave treatment as opposed to conventional heating treatments (see Example). Furthermore, this new process can yield new bioactive peptides presenting enhanced medicinal and/or nutraceutical benefits.

It will be appreciated that the hydrolyzing reaction is terminated in order to produce the protein hydrolysate containing the one or more isolated bioactive peptides. The person skilled in the art will know when it would be appropriate to terminate the hydrolysis reaction.

Proteolytic digestion of the microwave treated protein may be carried out generally by methods known to those skilled in the art. Suitable proteolytic enzymes include, but are not limited to, acid, neutral and alkaline proteases and peptidases, including serine endopeptidases, aspartate proteases, cystein proteases, or mixtures thereof. Many commercially available proteolytic enzymes can be used such as, for example, pepsin, trypsin and chymotrypsin, papain, alcalase and bacterial proteases. One of skill in the art can readily determine the appropriate digestion conditions for the particular proteolytic enzyme employed. In a preferred embodiment, the proteolytic enzyme used is selected from the group consisting of pepsin, trypsin, chymotrypsin and mixtures thereof. The hydrolyzing step or proteolytic digestion of the process of the present invention can be carried out sequentially with at least two different proteolytic enzymes. Typically, the proteolytic enzyme is used at a physiological enzyme to substrate ratio from about 1:20 to about 1:250.

As used in the context of the present invention, bioactive peptides are specific protein fragments that can have a positive impact on body functions or conditions and may ultimately influence health. For example, bioactive peptides may affect the major body systems as the cardiovascular, digestive, immune and nervous systems. The beneficial health effects may be classified as antimicrobial, antioxidative, antithrombotic, antihypertensive or immunomodulatory, to name a few. As known in the art, the activity of the bioactive peptides is based on their inherent amino acid composition and sequence. Bioactive peptides are inactive within the sequence of the parent protein and can be released, isolated or generated by any known method in the art, such as hydrolytic digestion.

As used herein, hydrolytic digestion is a catalytic decomposition of a chemical compound by the addition of specific enzymes.

As used herein, by a protein is meant any complex organic macromolecules composed of one or more chains of amino acids. A protein can be derived from food, such as milk, eggs, fungi, animal (meat) or vegetables, or any other sources.

As used in the context of the present invention, microwave irradiation, also referred to as “microwave heating”, “radio frequency” or “electronic” heating, refers to the process of applying microwaves (radiowaves with wavelengths typically between 300 MHz and 300 GHz) to a material which can result in heating of the material. Microwave irradiation can take place using any suitable equipment such as a microwave oven or the like. As known in the art, microwaves in themselves are not heat. Indeed, it is the material absorbing microwaves that converts the energy to heat such as by interaction between microwaves and polar molecules or ions. Microwave radiation is considered in the art to not have sufficient energy to break any chemical bonds (E=1 J/mole) and so be a non-ionizing form of radiation.

A hydrolysate, as used in the present invention, means the digestion mixture obtained after proteolytic or hydrolytic digestion or at least partial proteolytic digestion of proteins, such as food proteins or other bioactive health promoting proteins.

In a preferred embodiment, the protein is a food protein or a protein derived from a food ingredient. Preferably, the food protein is a milk protein such as, for example, a whey protein (e.g., β-lactoglobulin, α-lactalbumin, bovine serum albumin, lactoferrin, immunoglobulins, and glycomacropeptides) or casein or a caseinate fraction. In a most preferred embodiment, the whey protein is the β-lactoglobulin. In other embodiments, the protein is a protein fraction of milk or whey which contains a mixture of proteins. Suitable protein fractions include, but are not limited to, whey protein concentrate (35% to 90%), milk concentrate, milk protein concentrate, whey, reduced lactose whey, demineralized whey, or whey protein isolate. Preferably, the food protein being processed is by weight composed of 30%, 40%, 50%, 60%, 70%, 75%, 80%, 85%, 90%, 95%, 97%, or 98% of proteins. The method of the present invention may further be carried out by using milk, milk concentrate, skimmed milk, skimmed milk powders, milk protein concentrates, mixture of whey proteins and casein, isolated whey proteins fractions or isolated casein fractions, in addition to plant protein sources such as, but not limited to, soy (as, for example, soy protein concentrate or soy protein isolate), wheat and corn as well as egg (as, for example, albumin) proteins. The protein content can range from 0.01 to 35% or more depending on the extent of the protein's solubility in aqueous media and its thermal stability.

The protein to be processed by the method of the present invention is preferably dissolved or dispersed in aqueous media, i.e. it is a protein solution. However, once processed, it can be dried (e.g., spray dried or lyophilized) to minimize water activity. Furthermore, the protein to be processed by the method of the present invention can be in an aqueous media of pH of 2 to 12. In a preferred embodiment, the protein has at, or near neutral pH (e.g., within one or two pH units, i.e., in the range of 5 to 9) to enhance its solubility. In an alternative embodiment, the protein can be in a dry or gel-like form.

Preferably, the microwave treatment is an isothermal multi-cycle microwave treatment. In one embodiment, the microwave irradiation has wavelengths of ˜1 cm corresponding to a frequency of 2.45 GHz or 940 MHz and power adjusted between 1 to 6000 W, preferably between 20 and 6000 W and most preferably at 30 W. In an embodiment of the present invention, the protein or food protein is subjected to at least one cycle of microwave irradiation. The number of cycles may vary from about 1 to about 100. In a preferred embodiment, the protein or food protein is subjected to 1 to 10 cycles of microwave irradiation and more preferably to 4 cycles. In an embodiment, each cycle constitutes 10 seconds of irradiation with the power set to 15 W (5% of the total power). Preferably, each cycle time depends on the volume and the applied power of irradiation with the power set to 15 W-300 W (5%-100% of the total power) and the frequency from 0.3 GHz to 300 GHz. The irradiation is carried out from 0, 1 to 20 seconds, preferably between 1 to 10 seconds and most preferably 6 seconds. The person skilled in the art would know that the total irradiation time can range from second to minutes depending on the other settings. Further, a person skilled in the art will have no difficulty in selecting an appropriate wavelength value, number of cycles, power adjustment or exposure time in relation with the type of protein or food protein. In particular embodiments, the protein or food protein is subjected to temperatures of at least 30° C., at least 60° C., or at least 90° C. In a preferred embodiment, the protein or food protein is subjected to temperatures of about 30 to about 90° C. In a preferred embodiment, the microwave irradiation is carried out at a frequency of 2.45 GHz at a power of 30 W at a temperature of 30 to 90° C. However, other combinations of frequency, power and temperature are possible.

In another embodiment, the present invention relates to a process for treating a whey or soy protein before hydrolytic digestion (‘pre-treatment’), the process comprising exposing the whey or soy protein to at least one cycle of microwave irradiation to produce a microwave treated whey or soy protein containing one or more bioactive peptides. The microwave treated whey or soy protein can be further hydrolyzed with at least one proteolytic enzyme to release, isolate or generate the bioactive peptides. Instead of hydrolytic digestion, the pre-treatment can also be followed by any other suitable method to release, isolate or generate at least one of the one or more bioactive peptides.

The present invention also relates to a process for preparing one or more bioactive peptides from a protein, the process comprising the steps of: a) exposing a whey or soy protein in a solution to at least one cycle of microwave irradiation to produce a microwave treated whey or soy protein, b) hydrolyzing the microwave treated whey or soy protein with at least one proteolytic enzyme, and c) terminating said hydrolyzing step in order to release, isolate or generate one or more bioactive peptides. In a preferred embodiment, the whey protein is a β-lactoglobulin protein.

Using the process of the present invention in association with the β-lactoglobulin protein, the following two bioactive peptides have been identified: VLDTDYKYLL and TPPVDDEALEK. It has been demonstrated that these peptides have enhanced ACE activity, hypertension modulating and antioxidant properties. Therefore, an aspect of the present invention is directed to a peptide comprising an amino acid sequence of VLDTDYKYLL or TPPVDDEALEK. From another aspect, use of these peptides for modulating hypertension in a mammal or for inhibiting ACE activity in a mammal is also covered. The present invention also includes food products, supplements and other products including these peptides.

The present invention also relates to a pharmaceutical composition including one or more bioactive peptides produced by any of the processes of the present invention with a pharmaceutically acceptable carrier. In one embodiment, the bioactive peptide is VLDTDYKYLL or TPPVDDEALEK.

As known in the art, ACE raises blood pressure by converting angiotensin I released from angiotensinogen by renin into the potent vasoconstrictor angiotensin II. ACE also degrades vasodilative bradykinin and stimulates the release of aldosterone in the adrenal cortex which hormone is known to increase the reabsorption of sodium ions and water and the release (secretion) of potassium ions in the distal convoluted tubules of the kidneys. This increases blood volume and blood pressure. Consequently, ACE-inhibitors may exert an antihypertensive effect. ACE is an exopeptidase, which cleaves dipeptides from the C-terminal of various peptide substrates. ACE is an unusual zincmetallopeptidase, as it is activated by chloride and lacks in vitro substrate specificity. The peptides α-lactorphin and β-lactorphin are liberated during in vitro proteolysis of bovine whey proteins from α-lactalbumin and β-lactoglobulin, respectively, and their pharmacological activity at micromolar concentrations was observed to improve arterial function in spontaneously hypertensive rats. Presently, the inventors have demonstrated that whey and soy hydrolysates also show ACE-inhibitory activity after proteolysis with different digestive enzymes, and several active peptides have been identified. These results demonstrate the existence of several biologically active whey-derived peptides and hydrolysates. It will be understood that these findings can be exploited in the development of foods with special health claims (e.g. treatment of hypertension) as well as in identifying new applications of bioactive health promoting proteins in food.

From another aspect, the present invention relates to the use of the microwave treated protein containing bioactive peptides or the bioactive peptides produced by the process of the present invention for modulating hypertension and/or for inhibiting ACE activity in a mammal and/or for providing any other types of medical or health benefits.

The present invention also relates to a method for modulating hypertension and/or for inhibiting ACE activity and/or for providing any other types of medical or health benefits in a mammal comprising the administration of the microwave treated protein containing one or more bioactive peptides or the bioactive peptides.

The present invention also relates to an ACE-inhibitory and antioxidant peptide obtained by the process described in the present invention.

It will be understood that the proteins containing one ore more bioactive peptides and/or bioactive peptides and/or hydrolysate proteins produced by the method of the present invention could have numerous functions and uses in the medicinal and/or nutraceutical perspective as a source of peptides that have, for example, antihypertensive, antioxidant, immunomodulatory, opioid antagonist, opioid agonist, antithrombotic and antimicrobial properties.

Furthermore, the present invention also relates to any type of product or a food product comprising the microwave treated protein containing one or more bioactive peptides and/or the bioactive peptides produced by the processes of the present invention. The person skilled in the art will know how to incorporate such treated proteins or bioactive peptides to an edible product as food supplements, nutraceutical, nutritional food or nutritional product or dietary supplements.

A nutraceutical as used herein is a food that provides medical or health benefits, including the prevention and treatment of disease. As known in the art, a nutraceutical is a product produced from foods but sold in pills, powders, and other medicinal forms not generally associated with food. Such products may be formulated from isolated proteins, dietary supplements and specific components derived from processed foods. This definition also includes a bio-engineered designer vegetable food (e.g., rich in antioxidant ingredients), nutritional food or nutritional product, functional food, medicinal food or pharmafood.

A nutritional food or nutritional product is, as known in the art, a food or product in the form of, but not limited to, a health bar, health shake, yogurt or yogurt-based preparation, health drink, infant formula or a bakery product such as biscuit, cookie, muffin, bread, cereal, noodle, cracker, snack food or other similar forms of foods.

For the purposes of the described invention, a dietary supplement is defined as a product that bears or contains, for example, one or more of the following dietary ingredients: a vitamin, a mineral, an herb or other botanical, an amino acid, a dietary substance for use by man to supplement the diet by increasing the total daily intake of that substance, or a concentrate (e.g., a meal replacement or energy bar), metabolite, constituent, extract, or combinations of these ingredients.

### BRIEF DESCRIPTION OF THE DRAWINGS

Further aspects and advantages of the present invention will become better understood with reference to the description in association with the following drawings in which:

FIG. 1 shows IC50 values (mg mL⁻¹) for ACE inhibition by β-lactoglobulin hydrolysates obtained by pepsin hydrolysis of untreated, microwave-treated according to an embodiment of the present invention, and conventionally heated β-lactoglobulin (Example);

FIG. 2 shows IC50 values (mg mL⁻¹) for ACE inhibition by β-lactoglobulin hydrolysates obtained by trypsin hydrolysis of untreated, microwave-treated according to an embodiment of the present invention, and conventionally heated β-lactoglobulin (Example);

FIG. 3 shows IC50 values (mg mL⁻¹) for ACE inhibition by β-lactoglobulin hydrolysates obtained by chymotrypsin hydrolysis of untreated, microwave-treated according to an embodiment of the present invention, and conventionally heated β-lactoglobulin (Example);

FIG. 4 shows IC50 values (mg mL⁻¹) for ACE inhibition by β-lactoglobulin hydrolysates obtained by a two-stage hydrolysis with pepsin followed by trypsin and chymotrypsin of untreated, microwave-treated according to an embodiment of the present invention, and conventionally heated β-lactoglobulin (Example);

FIG. 5 (A) UV-chromatogram of the MW60 tryptic β-lactoglobulin hydrolysate; (B) Mass spectrum of the selected chromatographic peak in (A); (C) MS/MS spectrum of the doubly charged ion m/z 838.5 (Example);

FIG. 6 shows the degree of hydrolysis determined by the o-phthaldialdehyde method in enzymatic hydrolysates from β-Lg, according to an embodiment of the present invention (Example);

FIG. 7 shows the degree of hydrolysis determined by the o-phthaldialdehyde assay method as a function of time for the enzymatic hydrolysis of β-Lg by simulated gastric digestion, according to an embodiment of the present invention (Example);

FIG. 8 shows the antioxidant activity of the β-Lg hydrolysates obtained by pepsin and trypsin hydrolysis of untreated, microwave-treated according to an embodiment of the present invention, and conventionally heated β-Lg, determined by DPPH assay (Example); and

FIG. 9 shows the antioxidant activity of the β-Lg hydrolysates obtained by chymotrypsin and two-stage enzymatic hydrolysis of untreated, microwave-treated according to an embodiment of the present invention, and conventionally heated β-Lg, determined by DPPH assay (Example).

### DETAILED DESCRIPTION OF THE INVENTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including”, “comprising”, or “having”, “containing”, “involving” and variations thereof herein, is meant to encompass the items listed thereafter as well as, optionally, additional items. In the following description, the same numerical references refer to similar elements. In the drawings, like reference characters designate like or similar parts.

Broadly, the present invention provides a process for producing treated proteins which include bioactive peptides within their sequences and/or bioactive peptides from proteins or food proteins or proteins derived from food ingredient for nutraceutical, nutritional food, nutritional products or dietary supplement or food compositions with specific health claims or pharmaceutical compositions. Surprisingly, the inventors have found that the use of microwave treatment of proteins can produce microwave treated proteins having improved or enhanced health promoting properties. In this specific embodiment, the present invention is directed to a process for treating a protein before hydrolytic digestion comprising exposing the protein to at least one cycle of microwave irradiation to produce a microwave treated protein containing one or more bioactive peptides. In this embodiment, the microwave treatment of the present invention is followed by an enzymatic hydrolysis or proteolytic digestion by means of an in vitro digestion with enzymes currently employed in generating hydrolysate(s) in order to release one or more bioactive peptides. The inventors have further demonstrated that the bioactive peptides produced in accordance with the treatment by microwave heating followed by enzymatic hydrolysis are an improvement over the art in that the new peptides have, amongst other, enhanced ACE I inhibition and/or antioxidant properties and/or any known medicinal or health benefits over native protein sources. Furthermore, it will be understood that the proteins containing one or more bioactive peptides and/or hydrolysate proteins and/or bioactive peptides produced by the method of the present invention provide a non-pharmacological alternative for the prevention, control, modulation and/or treatment of numerous diseases or conditions known by the person skilled in the art. Furthermore, such proteins containing one or more bioactive peptides and/or hydrolysate proteins and/or bioactive peptides can also show, for example, enhanced antioxidant properties. Indeed, multi-cycle isothermal microwave treatment prior to enzymatic hydrolysis is not known to affect the peptide profile of the resulting hydrolysates.

In further detail and in order to produce a protein containing one or more bioactive peptides or bioactive peptides of the present invention, a protein or food protein or protein derived from food ingredient is selected and prepared in a solution, preferably an aqueous solution, at a suitable concentration. The concentration is between 0.01 to 35% depending on the solubility characteristics of the protein. In a preferred embodiment, the protein or food protein or protein derived from food ingredient is a β-lactoglobulin protein of a whey protein.

The protein solution is then subjected to a microwave treatment or irradiation. In a preferred embodiment, the microwave treatment is an isothermal multi-cycle microwave treatment. The microwave treatment is carried out at a frequency of between 300 MHz and 300 GHz with a power adjusted between 1 and 6000 W. The protein is subjected to a temperature of between about 30° C. to about 90° C. Preferably, the microwave irradiation is carried out at a frequency of 2.45 GHz or 940 MHz or other commercially available microwave systems. Preferably, the microwave irradiation is at a power of 20-6000, and most preferably 30 W. The protein or food protein or protein derived from food ingredient is exposed to the microwave irradiation for about 0.1 to 20 seconds, preferably for about 1 to 10 seconds and most preferably for 6 seconds and for 1 to 100 cycles, preferably 1 to 10 cycles and most preferably 4 cycles.

The microwave treated proteins treated are then proteolytically digested with at least one type of proteolytic enzyme in order to generate or produce a hydrolyzed protein product containing bioactive peptides. In a preferred embodiment, the proteolytic enzyme is pepsin, chymotrypsin, trypsin or a mixture thereof. Furthermore, it will be understood that the digestion step can be carried out sequentially with at least two different proteolytic enzymes. Preferably, the proteolytic enzyme is used at a physiological enzyme to substrate ratio from about 1:20 to about 1:250. If the resulting hydrolysate is to be used as an edible product such as a food or a food supplement, it may be desirable to stop the proteolytic digestion in order to retain some of the taste and functional properties of the food protein. The proteolytic digestion or hydrolyzing step is terminated by any suitable method known in the art as, for example, heat inactivation of the enzyme or adjustment of the pH of the digestion mixture away from the pH range of the enzyme activity.

In alternative embodiments, the above described process can be applied to other types of proteins such as those derived from soy, wheat, egg, corn, vegetable, microbial or animals.

The microwave treated protein containing one or more bioactive peptides along with the bioactive peptides produced by the proteolytic digestion of the microwave treated protein or food protein or protein derived from food ingredient can be used as an edible product or may be spray-dried to give a water-soluble powder for use as an edible product, as a pharmaceutical, as a food product, as food supplements, nutraceutical, nutritional food or nutritional product or dietary supplements, or the like.

The protein containing one or more bioactive peptides or bioactive peptides of the invention in liquid or powder form may be used to supplement beverages such as soft drinks, carbonated beverages, ready mix beverages, milk and milk beverages and their derivatives, and foods such as sauces, condiments, salad dressings, fruit juices, syrups, desserts (e.g. puddings, gelatin, icings, and fillings, baked goods and frozen desserts such as ice creams and sherbets), soft frozen products (e.g. soft frozen creams, soft frozen ice creams and yogurts, soft frozen toppings such as dairy or non-dairy whipped toppings), oil and emulsified products (e.g. shortening, margarine, mayonnaise, butter and salad dressings), candy and bar confections, cereal foods, and chewing gum table and the like. The proteins or hydrolysates of the present invention may also be formulated as pills, capsules, tablets, granules, powders, syrups, or suspensions. As such, the proteins or protein hydrolysates are admixed with a suitable carrier or excipient to facilitate processing into a particular shape or to improve palatability or solubility. A suitable carrier or excipient is a compound that is generally non-toxic and is commonly used to formulate compositions for animal or human consumption. The selection of a suitable carrier or excipient can be readily determined by one of skill in the art and can be dependent upon the form of the food protein or hydrolysates. Examples of suitable carriers and excipients include, but are not limited to, water, ethanol, glycerin, sodium citrate, calcium carbonate, calcium phosphate, starch (preferably potato or tapioca starch), alginic acid, certain complex silicates, sucrose, lactose, gelatin as well as high molecular weight polyethylene glycols, flavoring agents, coloring matter or dyes and, if so desired, emulsifying and/or suspending agents, and various combinations thereof. See, e.g., Remington: The Science and Practice of Pharmacy, Alfonso R. Gennaro, editor, 20th ed. Lippincott Williams & Wilkins: Philadelphia, Pa., 2000.

### EXAMPLES

The following examples demonstrate the present invention in greater details. These examples are not intended to limit the scope of the invention in any way.

### Example 1

### Materials and Methods

### β-Lactoglobulin Samples:

Bovine β-lactoglobulin (β-Lg) composed of mixtures of genetic variants, A and B, was obtained from Davisco Foods International (Eden Prairie, Minn., USA). The purity of the protein was confirmed by ESI-MS.

Two sets of β-Lg solutions were prepared in triplicate at a concentration of 5% (w/v) in H2O (pH ˜6.8). These two sets were employed in the microwave irradiation and conventional heating studies, respectively.

### Microwave Treatments:

The β-Lg solutions prepared as described above were subjected to microwave irradiation using a focused microwave Synthewave 402 (PROLABO, Fontenay-Sous-Bois, France), operating at a frequency of 2.45 GHz (λ=12 cm), with adjustable power between 15 and 300 W. A special equipment set-up was built in order to have very good temperature control in both the microwave and conventional thermal treatments. For purposes of comparison, β-Lg solutions were heated in a water bath to the same temperatures, times and cycles as targeted in the microwave experiments. The temperature of the solution was measured at the microwave cavity prior, during, and immediately after irradiation. The accuracy of the temperature measurements was within +0.5° C.

Samples of β-Lg that had been microwave or thermally-treated by conventional treatments at 40° C., 60° C. or 90° C., as well as native β-Lg were examined. These samples are designated MW40 and CH40, MW60 and CH60, and MW90 and CH90, respectively.

### Digestion of β-Lg:

The following enzymes were employed in these studies and were obtained from Sigma-Aldrich: pepsin (EC 3.4.23.1) from porcine gastric mucosa; α-chymotrypsin (EC 3.4.21.1) and trypsin (EC 3.4.21.4) from bovine pancreas.

In the enzymatic hydrolysis of microwave- and conventionally-heated β-Lg, control solutions were prepared under the same conditions but without the addition of enzyme. All experiments were performed in triplicate.

1. Pepsin Hydrolysis:

β-Lg (10 mg) was solubilized in 1 mL of 0.1 M HCl. The solution was then incubated at 37° C. and the pH adjusted to 2 using 1.0 M NaOH. The hydrolysis reaction was initiated by the addition of a pepsin solution (10 mg/mL) at an enzyme:substrate (E:S) ratio of 1:20. Following gentle stirring for 6 h, the reaction was stopped by heating the solution to 80° C. for 15 min, and the pH was then adjusted to 7.0.

2. Chymotrypsin and Trypsin Hydrolysis:

β-Lg (10 mg) was solubilized in 1 mL of 0.1 M sodium phosphate buffer (pH 8.0) and the resultant solution was incubated at 37° C. The hydrolysis reaction was initiated by the addition of a chymotrypsin or trypsin solution (10 mg/mL) at an E:S ratio of 1:100. Following gentle stirring for 4 h, the reaction was stopped in the same manner as for pepsin hydrolysis.

3. Two-Stage Hydrolysis (Simulated Gastrointestinal Digestion):

A two-stage hydrolysis process was carried out according to the method described by Vercruysse et al. (2005) whereby consecutive hydrolysis of β-Lg with pepsin, trypsin and α-chymotrypsin took place. First, the samples were acidified by lowering the pH to 2 with HCl (4 M), pepsin was added (E/S: 1:250), and the samples were incubated for 2 h at 37° C. to mimic digestion in the stomach. Second, incubation with trypsin (E/S: 1:250) and α-chymotrypsin (E/S: 1:250) at pH 6.5 (pH adjusted with 10 M NaOH) for 2.5 h at 37° C. was carried out to simulate digestion in the small intestine.

Aliquots were removed at 0, 60, 120, 240, and 360 min during the course of the trypsin and chymotrypsin hydrolysis, the pH was maintained by the addition of 2N NaOH:KOH (1:2) according to the pH-stat technique of Adler-Nissen (1977), while in the case of the pepsin hydrolysis, the pH was maintained by the addition of 1M NaOH hydrolysis. When the hydrolysis reaction was complete (confirmed by no further change in the pH of the reaction mixture), the reaction mixture was heated to 95° C. for 10 min, in a water bath, followed by cooling to room temperature. Samples were stored at −20° C. for subsequent analysis. All hydrolysis reactions were performed in triplicate.

### Measuring the Degree of Hydrolysis:

The extent of hydrolysis was assayed directly by quantification of cleaved peptide bonds using the o-phthaldialdehyde (OPA) spectrophotometric assay described by Church et al. (1985).

### ACE Inhibition:

ACE-inhibitory activity of β-Lg hydrolysates derived from the enzymatic hydrolysis of native, microwave-treated and conventionally heated β-Lg was measured by the spectrophotometric assay of Cushman and Cheung (1971) with some changes. A 0.3% (w/v) solution of hippuryl-L-histidyl-L-leucine (HHL) (Sigma H-1635) was prepared in 50 mM HEPES HCl buffer (Sigma H-7006) containing 300 mM NaCl (pH 8.3). A 200-μL aliquot of the HHL solution was mixed with 100 μL it of each hydrolysate (10 mg mL-1).

The reaction was initiated by addition of 50 μL of rabbit ACE (Sigma, EC 3.4.15.1) dissolved in cold deionized water at 0.33 U mL-1. Samples were incubated for 30 min at 37° C., and the reaction was then stopped by the addition of 0.25 mL of 1 M HCl. The hippuric acid liberated from the reaction was extracted from the solution into 2 mL of ethyl acetate by vortex mixing for 1 min. The sample was centrifuged (5000×g, 2 min) and 1 mL of the ethyl acetate layer was transferred into a clean tube and heated at 95° C. for 10 min to evaporate the solvent, re-dissolved in 3 mL of distilled water and mixed by inversion. The absorbance of the samples was measured spectrophotometrically at 228 nm. For each tested sample, a blank was prepared by adding 250 μl of HCL (1 M) before adding the enzyme. The reaction on ACE in the absence of inhibitor was carried out by replacing the protein hydrolysate by deionized distilled water. A blank was also prepared. The activity of each sample was tested in triplicate. The amount of hippuric acid liberated from the reaction in the absence of an inhibitor was defined as 100% ACE activity. Captopril, a synthetic ACE inhibitor, was used as a positive control (IC50=0.008 μM). The ACE activity (U mL-1) was calculated as:

$$ACE activity = \frac{( {A}_{228 nm}^{test} - {A}_{228 nm}^{blank} ) {aV}_{h}}{{V}_{e} Et ɛ} = \frac{( {A}_{228 nm}^{test} - {A}_{228 nm}^{blank} ) \times 2 \times 3}{9.8 \times 30 \times 0.91 \times 0.05} = 0.4485 ( {A}_{228 n m}^{test} - {A}_{228 nm}^{blank} )$$

where:
a is a conversion factor equal to 2, since the hippuric acid detected is only half of the total amount produced in the assay.
A_{228 nm}^{test} is the absorbance of the test solution at 228 nm,
A_{228 nm}^{blank} is the absorbance of the blank solution at 228 nm,
E is the extraction efficiency of ethyl acetate, and is equal to 0.91,
t is the duration of the assay (min) and is equal to 30,
Ve is the volume of enzyme added (mL) and is equal to 0.05,
Vh is the total volume of the hippuric acid solution (mL) and is equal to 3.0, and
ε is the millimolar extinction coefficient of hippuric acid at 228 nm, and is equal to 9.8.

Therefore, the ACE inhibitory activity expressed as a percentage is equal to ACE activity expressed as a percentage subtracted from 100. Inhibitory activity was calculated as the protein concentration (mg mL-1) needed to cause a 50% inhibition of the original ACE activity (IC50).

### Peptide Identification by Liquid Chromatography-Electrospray Ionization Mass Spectrometry and Tandem Mass Spectrometry:

Hydrolyzed β-Lg samples were dialyzed through a 30 kDa molecular weight cut-off, lyophilized and dissolved in 0.1% formic acid. The hydrolysates were subjected to RP-HPLC on a Widepore C18 column (250 mm×4.6 mm) (Bio-Rad, Richmond, Calif., USA). Operating conditions were: column at ambient temperature; flow rate, 0.8 mL min-1; injection volume, 5 μL; a binary gradient of solvent B (acetonitrile:0.1% formic acid) and solvent A (water:0.1% formic acid) was increased from 5 to 50% (A towards B) in 40 min. Injection volume was 5 μL. A flow of approximately 20 μL min-1 was directed into a Waters Micromass QTOF Ultima Global (Micromass; Manchester, UK) hybrid mass spectrometer equipped with a nanoflow electrospray source via the electrospray interface. Operating conditions were: positive ionization mode (+ESI) at 3.80 kV with a source temperature of 80° C. and desolvation temperature of 150° C. The TOF was operated at an acceleration voltage of 9.1 kV, a cone voltage of 100 V and collision energy of 10 eV (for MS survey). For the MS survey mass range, m/z was 400-1990, and for MS/MS, 50-1990, scanned continuously over the chromatographic run. Instrument control and data analysis were carried out by MassLynx v. 6.0 software (Waters Corporation, 2005).

Database analysis of the MS/MS data for sequence characterization was done using two software packages: ProteinLynx (Waters Corporation, 2005) and Mascot (Perkins et al., 1999).

### Statistical Analyses:

Experimental results were recorded as a mean±standard error. Data were analyzed using SAS for Windows (version 9.1) following an analysis of variance (ANOVA) one-way linear model. Mean comparisons were performed using the Duncan test, and the significance level of P≦0.05 was considered to indicate significance.

### Results:

### ACE Inhibition:

Enzymatic hydrolysates of bovine β-lactoglobulin subjected to microwave treatment at 40° C., 60° C. and 90° C. were analyzed for their ACE inhibition activity. The unhydrolyzed substrates gave very low (9%) ACE inhibition indices (IC₅₀ value of 8 mg mL⁻¹). Hydrolysis of β-lactoglobulin samples by pepsin, trypsin, and chymotrypsin and in a two-stage hydrolysis simulating gastrointestinal conditions resulted in higher IC₅₀ values, ranging from 0.36 to 0.99 mg mL⁻¹, IC₅₀ values for ACE inhibition by β-Lg hydrolysates obtained by enzymatic hydrolysis of untreated, microwave-treated and conventionally heated β-Lg samples are summarized in Table 1.

TABLE 1
IC50 values for ACE inhibition by β-Lg hydrolysates obtained by enzymatic
hydrolysis of untreated, microwave-treated and conventionally heated β-Lg samples

| | IC₅₀ (mg mL⁻¹)^{b} | | | |
|---|---|---|---|---|
| Sample | Pepsin | Trypsin | Chymotrypsin | Two-stage |
| pretreatment^{a} | hydrolysis | hydrolysis | hydrolysis | hydrolysis |
| Unheated β-Lg | 0.90 ± 0.01 b | 0.78 ± 0.03 c | 0.68 ± 0.01 c | 0.63 ± 0.01 c |
| CH 40 | 0.90 ± 0.02 b | 0.79 ± 0.01 c | 0.68 ± 0.04 c | 0.63 ± 0.03 c |
| MW40 | 0.86 ± 0.02bc | 0.74 ± 0.02 d | 0.57 ± 0.02 d | 0.51 ± 0.03 d |
| CH60 | 0.84 ± 0.02 c | 0.72 ± 0.01 d | 0.57 ± 0.04 d | 0.50 ± 0.04 d |
| MW60 | 0.80 ± 0.01 d | 0.65 ± 0.01 e | 0.43 ± 0.04 e | 0.36 ± 0.04 e |
| CH90 | 0.93 ± 0.03 a | 0.89 ± 0.02 b | 0.72 ± 0.03 b | 0.69 ± 0.02 b |
| MW90 | 0.99 ± 0.03 a | 0.98 ± 0.02 a | 0.75 ± 0.05 a | 0.74 ± 0.03 a |

^{b}Values presented are the average of two repetitions ± standard error. Column-wise [i.e., for each hydrolysing enzyme(s) treatment] values assigned the same letter show no significant difference from one another (p > 0.05).

The IC₅₀ values obtained for the protein hydrolysates conventionally pre-heated at 40° C. did not differ significantly from those of hydrolysates obtained from native β-Lg. In contrast, except in the case of pepsin, the IC₅₀ values of the hydrolysates microwave pretreated at 40° C. were significantly lower. In the case of samples pretreated at 60° C., hydrolysates microwave pretreated at 60° C. had significantly lower IC₅₀ values than hydrolysates conventionally heated at 60° C., which in turn had significantly lower values than native β-Lg hydrolysates for all enzyme treatments tested. Conventional or microwave heating of β-Lg to 90° C. resulted in higher IC₅₀ values than heating to 60° C., irrespective of the protease used. In the present study, however, the best ACE inhibition results were obtained from hydrolysates of β-Lg previously heated at 60° C., whereas hydrolysates obtained from unheated β-Lg or β-Lg heated to 90° C. showed significantly lower activity.

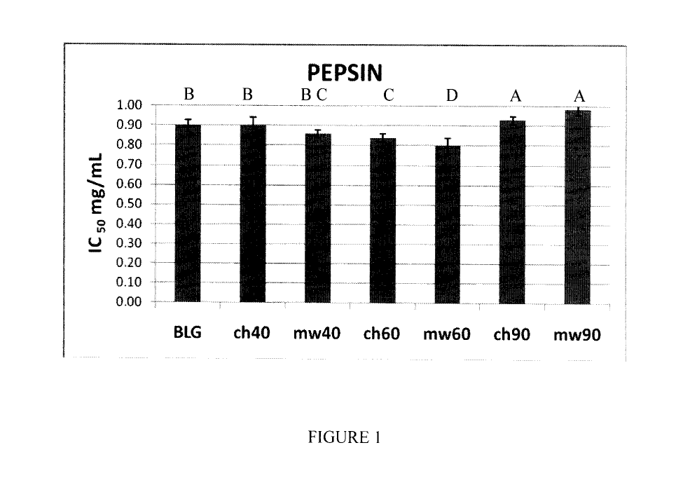

As shown in FIG. 1 (within each panel, treatments with the same letter have no significant differences (P>0.05)), the pepsin hydrolysates that were conventionally heated at 40° C. and the samples that were microwave treated at 40° C. showed no significant differences in ACE activity. However, the ACE activity of the microwave treated hydrolysate at 60° C. was significantly greater than that of the hydrolysate conventionally heated to 60° C. The results for microwave and conventionally heated treated β-Lg samples at 90° C. were not significantly different. Conventional or microwave heating of β-Lg to 90° C. resulted in significantly higher IC₅₀ values than heating to 60° C.

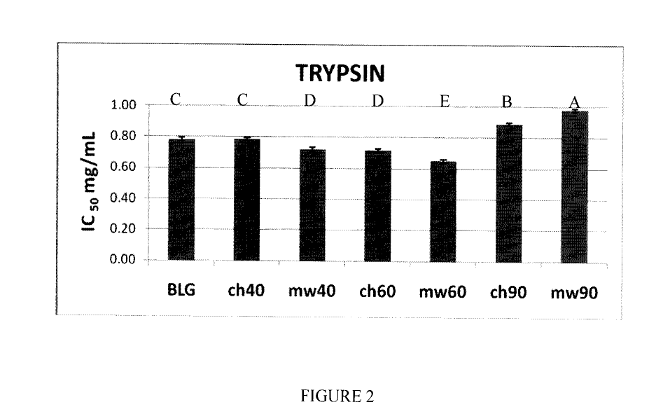

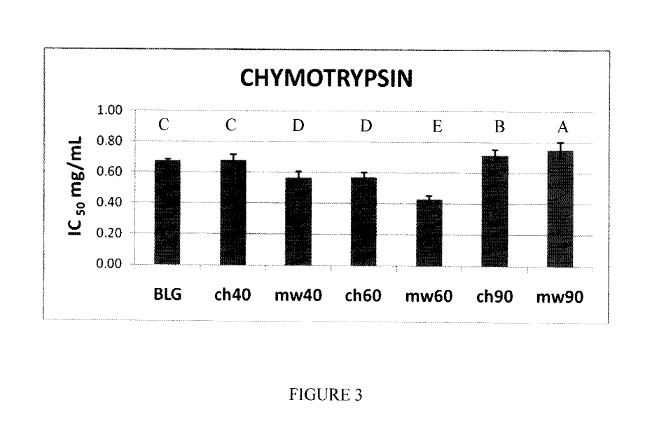

The trypsin and chymotrypsin hydrolysates, shown respectively in FIG. 2 (within each panel, treatments with the same letter have no significant differences (P>0.05)) and FIG. 3 (within each panel, treatments with the same letter have no significant differences (P>0.05)), showed significant differences between samples conventionally heated and microwave heated at all tested degrees.

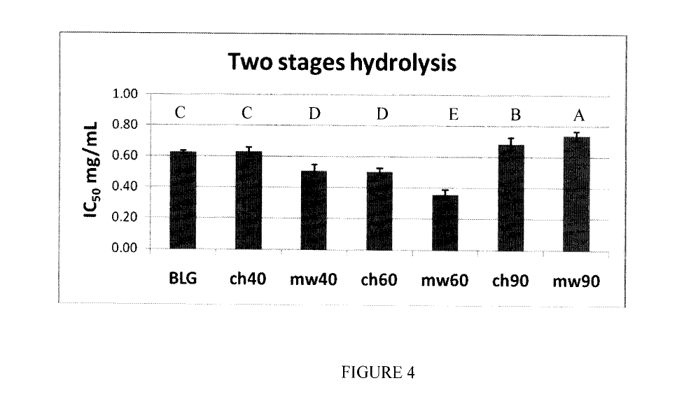

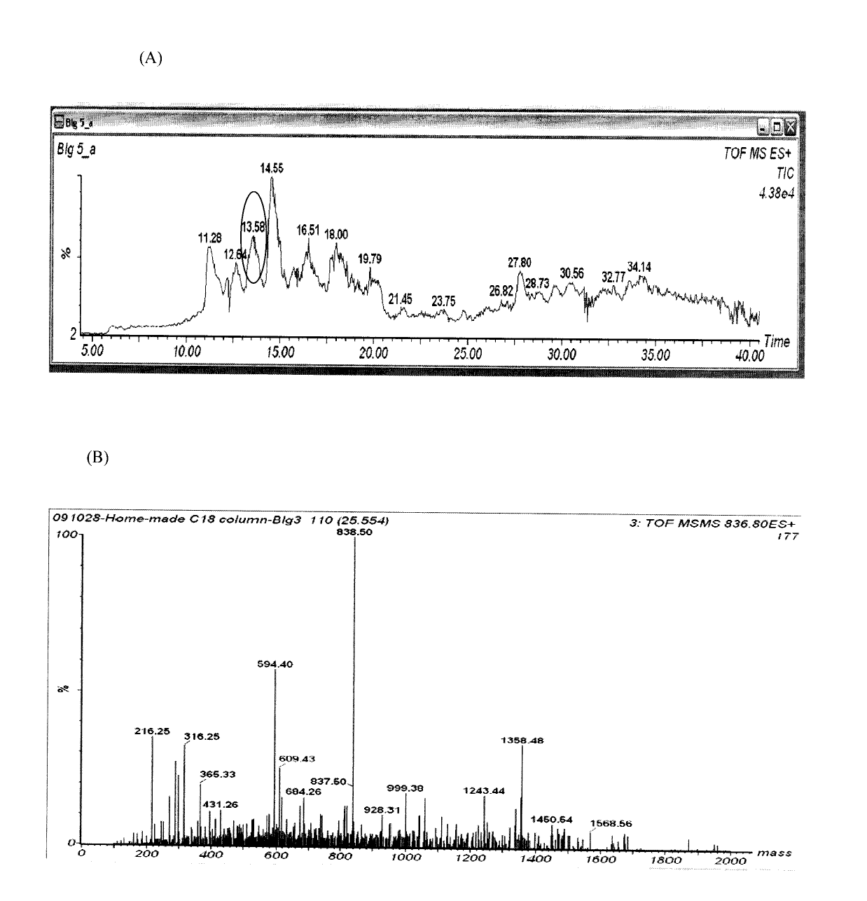

Similarly, the products of the two-stage hydrolysis (FIG. 4) of the β-Lg showed that the activity of hydrolysates of microwave-treated samples was significantly higher than that of the β-Lg hydrolysates of conventionally heated samples for all treatment temperatures (within each panel, treatments with the same letter have no significant differences (P>0.05)).

### Peptide Identification:

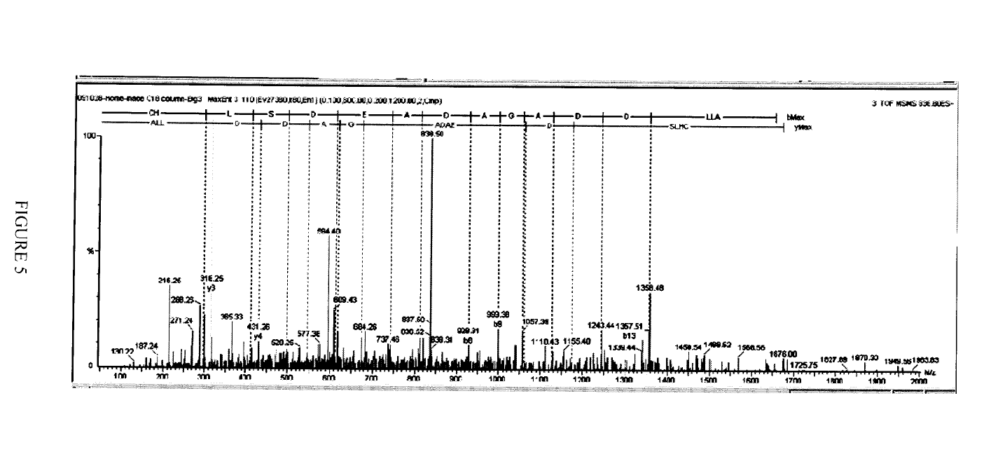

Hydrolysates bearing ACE inhibition activity derived from microwave and conventional heating were subjected to RP-HPLC-MS in order to identify the mass and primary sequence of the peptides as shown in FIG. 5.

The UV-chromatogram of a MW60 tryptic hydrolysate shows a complex peptidic profile, with 15 peptide peaks. The peptides identified by RP-HPLC-MS/MS of hydrolysates obtained from native, microwave-treated and conventionally heated β-Lg samples by proteolysis with chymotrypsin are given in Table 2. It is clear that chymotrypsin hydrolysis failed to produce any differences among the hydrolysates of these samples.

TABLE 2
Peptides identified by RP-HPLC-MS/MS in β-Lg chymotrypsin hydrolysates

| | Obs. | Calc. | Protein | | Sample | | | | | | |
|---|---|---|---|---|---|---|---|---|---|---|---|
| | Mass | mass | fragment | Sequence | β-Lg | CH40 | MW40 | CH60 | MW60 | CH90 | MW90 |
| 1 | 600.3 | 600.4 | 123-127 | VRTPT | + | + | + | + | + | + | + |
| 2 | 675.4 | 675.3 | 25-31 | AASDISL | + | + | + | + | + | + | + |
| 3 | 700.4 | 700.34 | 33-39 | DAQSAPL | + | + | + | + | + | + | + |
| 4 | 715.3 | 715.39 | 88-93 | NENKVL | + | + | + | + | + | + | + |
| 5 | 1119.4 | 1119.2 | 33.42 | DAQSAPLRVY | + | + | + | + | + | + | + |
| 6 | 813.3 | 813.42 | 32-39 | LDAQSAPL | + | + | + | + | + | + | + |
| 7 | 558.6 | 558.32 | 141-149 | KALPM | + | + | + | + | + | + | + |

The representative LC-ESI-MS-MS spectra for hydrolysates obtained from native, microwave-treated and conventionally heated β-Lg samples by proteolysis with trypsin are summarized in Table 3. The results show unique peptides associated with microwave-treated samples. For example, m/z 1245.6 assigned to f(125-135) was identified only in the MW60 hydrolysate, while m/z 2708.4 assigned to f (15-40) was identified in both the MW90 and CH90 hydrolysates.

TABLE 3
Peptides identified by RP-HPLC-MS/MS in β-Lg trypsin hydrolysates

| | Obs. | Calc. | Protein | | Sample | | | | | | |
|---|---|---|---|---|---|---|---|---|---|---|---|
| | Mass | mass | fragment | Sequence | β-Lg | CH40 | MW40 | CH60 | MW60 | CH90 | MW90 |
| 1 | 674.3 | 674.4 | 78-83 | IPAVFK | + | + | + | + | + | + | + |
| 2 | 673.4 | 673.4 | 9-14 | GLDIQK | + | + | + | + | + | + | + |
| 3 | 1452.4 | 1452.7 | 48-60 | PTPEGDLEILLQK | + | + | + | + | + | + | + |
| 4 | 573.4 | 573.4 | 71-75 | IIAEK | + | + | + | + | + | + | + |
| 5 | 916.5 | 916.5 | 84-91 | VLVLDTDYKK | + | + | + | + | + | + | + |
| 6 | 2675.2 | 2675.2 | 102-124 | YLL...LVR | + | + | + | + | + | + | + |
| 7 | 836.77 | 837.5 | 142-148 | ALPMHIR | + | + | + | + | + | + | + |
| 8 | 409.2 | 409.2 | 136-138 | FDK | + | + | + | + | + | + | + |
| 9 | 331.2 | 331.2 | 139-141 | ALK | + | + | + | + | + | + | + |
| 10 | 2708.4 | 2707.4 | 15-40 | VAG......LR | − | − | − | − | − | + | + |
| 11 | 1245.5 | 1245.6 | 125-135 | TPPVDDEALEK | − | − | − | − | + | − | − |

The representative LC-ESI-MS-MS spectra for hydrolysates obtained from native, microwave-treated and conventionally heated β-Lg by combined proteolysis with pepsin, trypsin and chymotrypsin, simulating gastrointestinal digestion are shown in Table 4. A notable number of peptides were released by this two-stage hydrolysis. They included all the peptides resulting from the action of trypsin or chymotrypsin individually except the peptide fragment with m/z 2675.2, detected in trypsin hydrolysates, which may have been further hydrolyzed by other enzymes to lower molecular weight fragments. Among the peptides released, m/z 2708.4 assigned to f(15-40), which was previously detected in trypsin hydrolysates, m/z 973 assigned to f(109-117) and m/z 751 (unassigned fragment), were identified only in the MW90 and CH90 hydrolysates, while m/z 1069.7 assigned to f(32-41), m/z 1108.4 assigned to f(125-134) and m/z 1109 (unassigned fragment) were identified only in the MW60, MW90 and CH90 hydrolysates. Three m/z peaks were unique to the MW60 hydrolysate: m/z 1370 assigned to f(94-104), m/z 1245.6 assigned to f(125-135) and m/z 1147 (unassigned fragment). Two m/z peaks were unique to the MW90 hydrolysate: 774.4 assigned to f(76-82) and 761.0 (unassigned fragment). Finally, m/z 855.5 assigned to f(46-53) was found only in the MW60 and MW90 hydrolysates.

TABLE 4
Peptides identified by RP-HPLC-MS/MS in β-Lg hydrolysates from two-
stage enzymatic hydrolysis

| | Obs. | Calc. | Protein | | Sample | | | | | | |
|---|---|---|---|---|---|---|---|---|---|---|---|
| | Mass | mass | fragment | Sequence | B-Lg | CH40 | MW40 | CH60 | MW60 | CH90 | MW90 |
| 1 | 600.3 | 600.4 | 123-127 | VRTPT | + | + | + | + | + | + | + |
| 2 | 674.3 | 674.4 | 78-83 | IPAVFK | + | + | + | + | + | + | + |
| 3 | 673.4 | 673.4 | 9-14 | GLDIQK | + | + | + | + | + | + | + |
| 4 | 675.3 | 675.3 | 25-31 | AASDISL | + | + | + | + | + | + | + |
| 5 | 700.4 | 700.34 | 33-39 | DAQSAPL | + | + | + | + | + | + | + |
| 6 | 715.39 | 715.39 | 88-93 | NENKVL | + | + | + | + | + | + | + |
| 7 | 753.35 | 753.35 | 95-100 | LDTDYK | + | + | + | + | + | + | + |
| 8 | 573.7 | 573.4 | 58-61 | LQKW | + | + | + | + | + | + | + |
| 9 | 1119.2 | 1119.2 | 33-42 | DAQSAPLRVY | + | + | + | + | + | + | + |
| 10 | 813.42 | 813.42 | 32-39 | LDAQSAPL | + | + | + | + | + | + | + |
| 11 | 558.32 | 558.32 | 141-145 | KALPM | + | + | + | + | + | + | + |
| 12 | 695.33 | 695.33 | 15-20 | VAGTWY (ace) | + | + | + | + | + | + | + |
| 13 | 828.3 | 828.3 | 87-93 | LNENKVL | + | + | + | + | + | + | + |
| 14 | 1040.6 | 1040.5 | 20-29 | VEELKPTPE | + | + | + | + | + | + | + |
| 15 | 1452.4 | 452.7 | 48-60 | PTPEGDLEILLQK | + | + | + | + | + | + | + |
| 16 | 573.7 | 573.4 | 71-75 | IIAEK | + | + | + | + | + | + | + |
| 17 | 916.4 | 916.5 | 84-91 | VLVLDTDYKK | + | + | + | + | + | + | + |
| 18 | 782.7 | 782.4 | 15-21 | VAGTWYS | + | + | + | + | + | + | + |
| 19 | 836.77 | 838.5 | 142-148 | ALPMHIR | + | + | + | + | + | + | + |
| 20 | 409.2 | 409.2 | 136-138 | FDK | + | + | + | + | + | + | + |
| 21 | 331.2 | 331.2 | 139-141 | ALK | + | + | + | + | + | + | + |
| 22 | 626.4 | 625.38 | 146-150 | HIRLS | − | − | − | + | + | + | + |
| 23 | 931.4 | 931.1 | 96-102 | DTDYKKY | + | + | + | + | + | + | + |
| 24 | 2708.4 | 2707.4 | 15-40 | VAG......LVR | − | − | − | − | − | + | + |
| 25 | 973.0 | 973.44 | 109-117 | NSAEPEQSL | − | − | − | − | − | + | + |
| 26 | 751 | | | n/a | − | − | − | − | − | + | + |
| 27 | 1374 | 1370 | 94-104 | VLDTDYKYLL | − | − | − | − | + | − | − |
| 28 | 1245.4 | 1245.6 | 125-135 | TPEVDDEALEK | − | − | − | − | + | − | − |
| 29 | 1147 | | | n/a | − | − | − | − | + | − | − |
| 30 | 804.4 | 804.44 | 36-42 | SAPLRVY | − | − | − | − | + | + | + |
| 31 | 1071.5 | 1069.7 | 32-41 | LDAQSAPLRV | − | − | − | − | + | + | + |
| 32 | 1109 | | | n/a | − | − | − | − | + | + | + |
| 33 | 1108.4 | 1108.4 | 125-134 | TPPVDDEALE | − | − | − | − | + | + | + |
| 34 | 775 | 774.4 | 76-82 | TKIPAVF | − | − | − | − | − | + | − |
| 35 | 761 | | | n/a | − | − | − | − | − | + | − |
| 36 | 856.0 | 855.5 | 46-53 | LKPTPEGD | − | − | − | − | + | − | + |

Many of the peptides listed in Tables 3 and 4 were previously described to have an ACE inhibition activity.

### Hydrolysis of β-Lg:

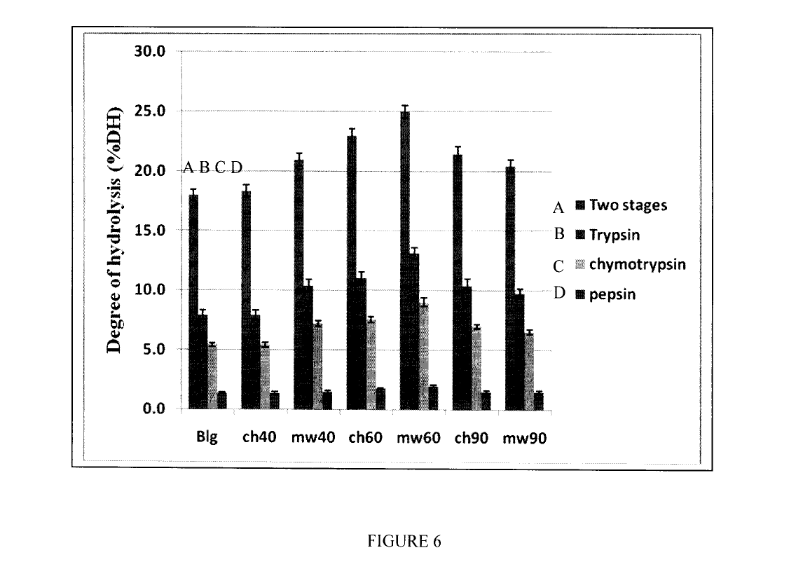

The extent of β-Lg hydrolysis at 37° C. by pepsin, trypsin, and chymotrypsin and by a simulated gastrointestinal enzymatic hydrolysis was determined during 6 hours of hydrolysis, in 30-min increments, by measuring the amount of α-amino groups released in the enzymatic reaction, using the o-phthaldialdehyde (OPA) reaction. Table 5 and FIG. 6 (error bars represent SE of triplicate experiments) show the degree of hydrolysis of β-Lg after each enzyme treatment. The values are the mean and standard error of triplicate experiments. Simulated gastrointestinal enzymatic hydrolysis was the most effective β-Lg hydrolysis method, followed by trypsin, chymotrypsin and pepsin hydrolysis, respectively, irrespective of the sample treatment (see Table 5 and FIG. 6).

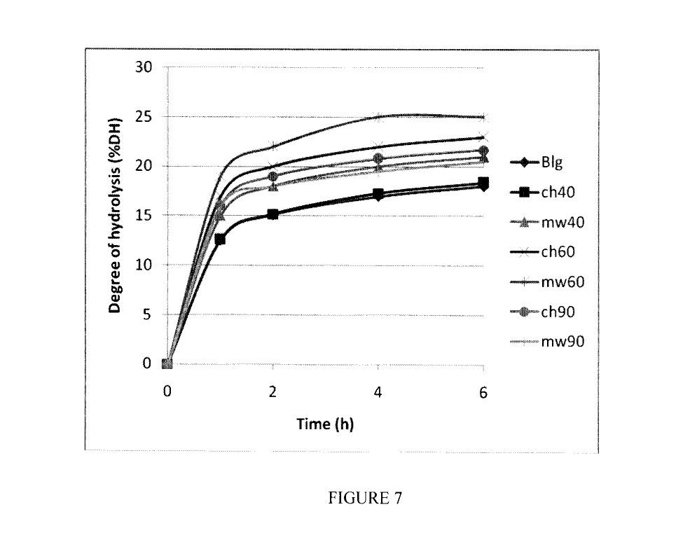

A plot of the degree of hydrolysis of β-Lg in a two-stage enzymatic digestion as a function of time (FIG. 7) shows that the ultimate extent of hydrolysis ranged from 18% in the case of native fβ-Lg to 25% in the case of the samples subjected to the MW60 pretreatment. Both microwave and conventional heat treatments of β-Lg significantly (P≦0.05) enhanced hydrolysis when compared to the control, with one exception: there was no significant difference (P>0.05) between the degree of hydrolysis following the CH40 treatment and that of the native β-Lg. Other conditions being the same, the degree of hydrolysis of β-Lg was significantly enhanced (P≦0.05) when digestion took place after microwave as opposed to conventional heating treatments.

TABLE 5
Degree of hydrolysis in enzymatic hydrolysates from
β-Lg, as determined by the o-phthaldialdehyde method.

| | Degree of Hydrolysis (%)^{b} | | | |
|---|---|---|---|---|
| Sample | | | | Two-stage |
| pretreatment^{a} | Pepsin | Trypsin | Chymotrypsin | hydrolysis |
| Untreated β-Lg | 1.4 ± 0.05 | 7.9 ± 0.38 | 5.4 ± 0.15 | 18.0 ± 0.49 |
| CH40 | 1.4 ± 0.11 | 7.9 ± 0.41 | 5.4 ± 0.21 | 18.4 ± 0.50 |
| MW40 | 1.5 ± 0.15 | 10.4 ± 0.49 | 7.2 ± 0.23 | 21.0 ± 0.55 |
| CH60 | 1.8 ± 0.05 | 11.1 ± 0.51 | 7.6 ± 0.22 | 23.0 ± 0.56 |
| MW60 | 2.0 ± 0.10 | 13.1 ± 0.48 | 9.0 ± 0.35 | 25.0 ± 0.54 |
| CH90 | 1.5 ± 0.15 | 10.4 ± 0.28 | 7.0 ± 0.19 | 21.5 ± 0.59 |
| MW90 | 1.5 ± 0.17 | 9.8 ± 0.18 | 6.5 ± 0.21 | 20.5 ± 0.49 |

^{b}Values are means ± SE of triplicate experiments.

For both the MW and the CH samples, those treated at 60° C. showed a significantly (P≦0.05) higher degree of hydrolysis than those treated at 90° C.; in turn, the latter showed significantly greater hydrolysis than native β-Lg or samples treated at 40° C. The decrease in the degree of hydrolysis following treatment at 90° C. relative to 60° C., which was more pronounced in the case of the microwave-treated samples, may be attributed to the protein aggregation that occurred at 90° C., rendering the protein less accessible to the enzyme action.

Similar trends were obtained for hydrolysis of β-Lg by trypsin and chymotrypsin, although the degrees of hydrolysis were uniformly lower than the corresponding values for the two-stage hydrolysis. For example, the greatest degrees of hydrolysis, which again occurred in the MW60 samples, were 13.1% and 9.0% with trypsin and chymotrypsin, respectively, while the values obtained for hydrolysis of untreated β-Lg with trypsin and chymotrypsin were 7.9% and 5.4%, respectively. In the case of hydrolysis by pepsin, the degree of hydrolysis of β-Lg was very limited for all samples, ranging from 1.4% for untreated β-Lg to 1.8% and 2% for the CH60 and MW60 samples, respectively, with no significant differences (P>0.05) being observed among any of the other pepsin hydrolysates.

### Antioxidant Activity:

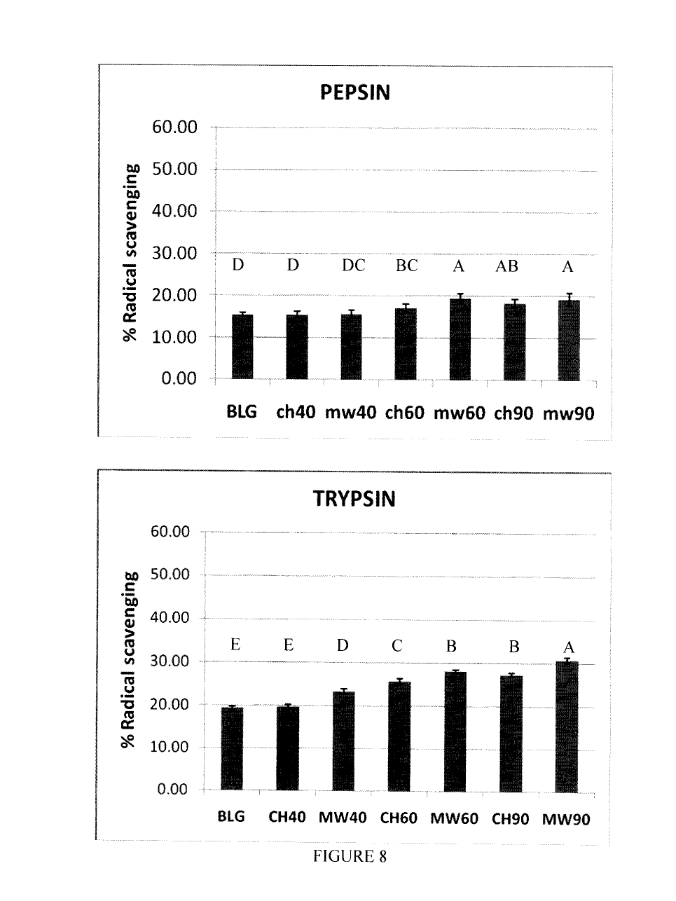

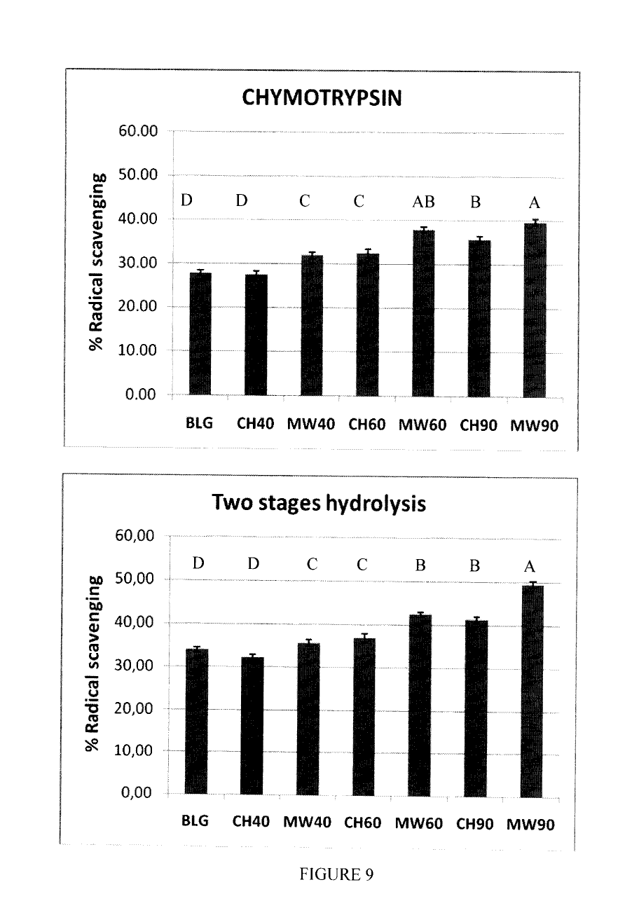

The antioxidant activity of β-Lg hydrolysates was evaluated in vitro using a radical scavenging assay based on the reduction of alcohol-stable DPPH by a free-radical-scavenging antioxidant. The reduction of DPPH was quantified spectrophotometrically by a decrease in absorbance. Vitamin C has a comparable radical scavenging activity, as has been previously reported. Table 6 and FIGS. 8 (within each panel, treatments with the same letter have no significant differences (P>0.05)) and 9 (within each panel, treatments with the same letter have no significant differences (P>0.05)) show the antioxidant activity determined by the DPPH assay for the β-Lg hydrolysates at a concentration of 5 mg/mL. The values are expressed as means of % radical scavenging ±SE based on three repetitions. Significant differences were determined by one-way ANOVA (P<0.05) and are denoted by letters a-e.

There were significant differences of % radical scavenging among all the enzyme treatments (Table 6). In the case of native β-Lg and β-Lg treated at 90° C. by microwave irradiation, the products of the two-stage hydrolysis showed the highest radical scavenging activity, 33.9% and 49.3%, respectively, followed by 27.7% and 39.6% for chymotrypsin hydrolysates, 19.3% and 28.2% for trypsin hydrolysates, and 15.5% and 19.3% for pepsin hydrolysates. For all enzymatic treatments, there was no significant difference in radical scavenging activity between the native β-Lg and the CH40 treatment. The antioxidant activity increased with the temperature at which the β-Lg samples had been treated and the highest activity was found in the hydrolysates obtained from β-Lg heated to 90° C.

The pepsin hydrolysates of the CH40 and MW40 samples showed no significant differences in antioxidant activity. However, the antioxidant activity of the MW60 hydrolysate was significantly greater than that of the CH60 hydrolysate. The results for samples treated at 90° C. were not significantly different from those for samples treated at 60° C. Finally, for trypsin, chymotrypsin hydrolysates and the products of the two-stage hydrolysis, the antioxidant activity of hydrolysates of microwave-treated samples was significantly higher than that of hydrolysates of conventionally heated samples for all treatment temperatures.

TABLE 6
Antioxidant activity of the β-Lg hydrolysates determined by DPPH assay

| | Radical scavening (%)^{b} | | | |
|---|---|---|---|---|
| Sample | Pepsin | Trypsin | Chymotrypsin | Two-stage |
| pretreatment^{a} | hydrolysis | hydrolysis | hydrolysis | hydrolysis |
| Unheated β-Lg | 15.52 ± 0.51d | 19.35 ± 0.72 e | 27.75 ± 1.2 d | 33.91 ± 0.62 d |
| CH 40 | 15.52 ± 1.22 d | 19.76 ± 0.63 e | 27.57 ± 0.8 d | 32.18 ± 0.59 d |
| MW40 | 15.69 ± 0.75 cd | 23.39 ± 0.9 d | 31.89 ± 1.2 c | 35.65 ± 0.71 c |
| CH60 | 17.24 ± 0.65 bc | 25.81 ± 0.72 c | 32.43 ± 1.4 c | 36.84 ± 0.70 c |
| MW60 | 19.54 ± 1.0 a | 28.11 ± 0.99 b | 37.93 ± 1.2 ab | 42.33 ± 0.75 b |
| CH90 | 18.39 ± 1.4 ab | 28.42 ± 0.92 b | 35.68 ± 1.3 b | 41.21 ± 0.71 b |
| MW90 | 19.37 ± 0.9 a | 30.83 ± 0.93 a | 39.64 ± 1.5 a | 49.43 ± 1.00 a |

^{b}Mean % radical scavenging ± SE (n = 3). Column-wise [i.e., for each hydrolyzing enzyme(s) treatment] values assigned the same letter show no significant difference from one another (P > 0.05). Significant differences determined by one-way ANOVA

The Examples show that microwave heating of aqueous solutions of β-Lg result in a greater population of unfolded β-Lg than conventional heating at the same temperature.

The inventors have demonstrated that the ACE activity was greatest in hydrolysates obtained from β-Lg that had been microwave heated to 60° C. Mass-spectrometric analysis of hydrolysates of β-Lg that had received prior microwave treatment revealed the presence of several unique peptides as well as higher amounts of peptides known to have high ACE inhibition activity than were present in the hydrolysates obtained from conventionally heated β-Lg.

Microwave treatment of β-Lg at 60° C. followed by conventional enzymatic hydrolysis (a two-stage enzymatic hydrolysis) yielded two completely new peptides, VLDTDYKYLL and TPPVDDEALEK. Two stage enzymatic hydrolysis e.g. pepsin followed by trypsin and chymotrypsin gave the best results. Accordingly, the microwave treated proteins and the protein hydrolysates formed from protein or food protein with the process of the present invention which implies a microwave treatment before enzymatic hydrolysis are suitable for use in nutraceutical, nutritional food or nutritional product, dietary supplement compositions and pharmaceutical compositions.

In other words, the inventors have demonstrated that hydrolysates of microwave treated whey protein at 60° C. result in two new peptides compared to conventional heating and the control (non-treated sample) (m/z 493, m/z 875 and m/z 804 in addition to two peaks which are m/z 701 and m/z 477).

### Example 2

The same methodology was applied to whey protein isolate (WPI) and soy protein isolate (SPI). The degree of hydrolysis and ACE inhibition of two stages enzymatic hydrolysates were evaluated as mentioned above.

TABLE 7
Degree of hydrolysis (DH %) and ACE inhibition
of two stages enzymatic hydrolysates.

| Sample | Treatment | DH % | AC50 |
|---|---|---|---|
| WPI | Non-treated | 18.8 ± 0.2 | 0.74 ± 0.01 |
| | Conventionally heated | 22.2 ± 0.3 | 0.73 ± 0.01 |
| | Microwaved | 23.7 ± 0.3 | 0.69 ± 0.01 |
| SPI | Non-treated | 19.7 ± 0.2 | 0.70 ± 0.01 |
| | Conventionally heated | 22.8 ± 0.2 | 0.66 ± 0.01 |
| | Microwaved | 25.4 ± 0.2 | 0.62 ± 0.01 |

The inventors have demonstrated that microwave effect is confirmed using the whey protein isolate (WPI) and soy protein isolate (SPI). Hydrolysates pre-treated with microwave irradiation showed higher degree of hydrolysis and higher ACE inhibition activity compared with conventionally heated and non-treated proteins.

### REFERENCES

Adler-Nissen (1977). Enzymatic hydrolysis of food proteins. Process Biochem. 12 (6) 18-23.32.
Church, F. C., Porter, D. H., Catignani, G. L. and Swaiscoogood, H. E. (1985). An o-phthalaldehyde spectrophotometric assay for proteinases. Anal. Biochem. 146: 343-348.
Cushman, D. W. and Cheung, H. S. (1971). Spectrophotometric assay and properties of the angiotensin-converting enzyme of rabbit lung. Biochem. Pharmacol. 20: 1637-1648.
Perkins, D. N., Pappin, D. J. C., Creasy, D. M. and Cottrell, J. S. (1999). Probability-based protein identification by searching databases using mass spectrometry data. Eletrophoresis 20: 3551-3567.
Vercruysse, L., Smagghe, G., Herregods, G. and Van Camp, J. (2005). ACE inhibitory activity in enzymatic hydrolysates of insect protein. J. Agric. Food Chem. 53: 5207-5211.

It should be appreciated that the invention is not limited to the particular embodiments described and illustrated herein but could includes all modifications and variations falling within the scope of the invention as defined in the appended claims.

1. A process for treating a protein before hydrolytic digestion, the process comprising exposing the protein to at least one cycle of microwave irradiation to produce a microwave treated protein containing one or more bioactive peptides.

2. The process of claim 1, further comprising hydrolytic digestion of the microwave treated protein with at least one proteolytic enzyme to release at least one of the one or more bioactive peptides.

3. The process of claim 2, further comprising terminating said hydrolytic digestion in order to release the one or more bioactive peptides.

4. The process of claim 1, wherein the protein is a food protein or a protein derived from food ingredient.

5. The process of claim 4, wherein the food protein is a whey protein or a soy protein.

6. The process of claim 5, wherein the whey protein is β-lactoglobulin or α-lactalbumin.

7. (canceled)

8. The process of claim 1, wherein the bioactive peptide is VLDTDYKYLL (SEQ ID NO:1) or TPPVDDEALEK (SEQ ID NO:2).

9. (canceled)

10. The process of claim 1, wherein the microwave irradiation is carried out at a frequency of between about 0.3 to about 300 GHz, preferably 2.45 GHz.

11. The process of claim 1, wherein the microwave irradiation is carried out at a power of between about 1 and 6000 W, preferably between 20 and 6000 W, and most preferably at 30 W.

12. The process of claim 1, wherein the protein is subjected to a temperature of at least 30° C., preferably between about 30 to 90° C., during the microwave irradiation.

13. The process of claim 1, wherein the protein is exposed to the microwave irradiation for from about 1 to about 100 cycles, preferably for about 1 to about 10 cycles, of about 0.1 to about 20 seconds.

14. (canceled)

15. The process of claim 2, wherein the proteolytic enzyme is a pepsin, trypsin, chymotrypsin or mixtures thereof.

16. The process of claim 12, wherein said hydrolytic digestion is carried out sequentially with at least two different proteolytic enzymes.

17. The process of claim 13, wherein the proteolytic enzyme is used at a ratio from 1:20 to 1:250.

18. The process of claim 1, wherein the concentration of the protein is in the range of about 1 to about 35%, preferably about 1 to about 10%, most preferably about 5%.

19-31. (canceled)

32. A method for modulating hypertension in a mammal comprising administering to said mammal the microwave treated protein of claim 1.

33. (canceled)

34. A method for inhibiting ACE activity in a mammal comprising administering to said mammal the microwave treated protein of claim 1.

35-42. (canceled)
